(19) United States
(12) Patent Application Publication
(10) Pub. No.: US 2025/0230219 A2
(43) Pub. Date: Jul. 17, 2025

(54) METHOD OF PRODUCING COLLAGEN

(71) Applicant: HOLISTA COLLTECH LIMITED, Subiaco, Western Australia (AU)

(72) Inventor: M Rajendran V MARNICKAVASAGAR, Petaling Jaya (MY)

(73) Assignee: HOLISTA COLLTECH LIMITED, Subiaco, Western Australia (AU)

(21) Appl. No.: 18/208,249

(22) Filed: Jun. 9, 2023

(30) Foreign Application Priority Data
May 19, 2023 (MY) PI 2023002999

(51) Int. Cl. C07K 14/78; B01D 21/26; C07K 1/30; C07K 1/34; C12N 9/50; C12N 9/58

(52) U.S. Cl. CPC C07K 14/78; B01D 21/262; C07K 1/303; C07K 1/34; C12N 9/58; C12N 9/63; C12Y 304/22031; C12Y 304/23

(57) ABSTRACT

The present disclosure relates to a method of producing collagen from an animal and more particularly, the present invention relates to a method for producing collagen by applying an acid and metal fluoride combination to an animal skin.

### FIELD OF THE INVENTION

The present disclosure relates to a method of producing collagen from an animal skin. More particularly, the present invention relates to a method for producing collagen by applying an acid to an animal skin.

### BACKGROUND OF THE INVENTION

Collagen is a structural protein that is a component of the extracellular matrix (ECM) of various connective tissues such as skin, bones, cartilage and tendon found in animal organisms where it provides the fundamental structural support. Collagen is the most abundant structural protein in all animals.

There exist at least twelve genetically distinct types of collagens. The most familiar, type I, consists of three polypeptide chains. Two chains are identical and are called a1 and the third is called a2. Type I collagen forms the major portion of the collagen of both soft (skin, tendon) and hard (bone and dentine) connective tissue. Type II collagen is the major collagen of cartilage and is composed of three a, 1 chain. Type III collagen is composed of three a1 chains and is found in blood vessels, wounds, and certain tumours.

Naturally synthesized collagen molecules consist of three long helicoidal chains of amino acid residues with nonhelical terminals at both ends. At least 46 unique polypeptide chains have been found in collagens of various animals. (See, Matinong, A. M. E.; Chisti, Y.; Pickering, K. L.; Haverkamp, R. G. collagen extraction from animal skin. Biology 2022, 11, 905).

The commercially available collagens are often produced from pigskin, cattle bones, bovine hide, and several other sources (Khong et al., 2018). The method of producing commercial collagen generally involves pretreatment and extraction process. The pre-treatment process can be characterized by acid pre-treatment, alkaline pre-treatment, and skin specific pre-treatment. Followed by pre-treatment process is the extraction which involves acid or alkali hydrolysis, salt stabilization, enzyme hydrolysis, ultrasound assisted extraction and microwave assisted extraction.

For instance, U.S. Ser. No. 09/357,908 discloses about collagen extracted and produced from a raw fish skin through the steps: a salt admixing step for admixing a salt (e.g. NaCl, KCl or the like) with a raw fish skin so as to allow non-collagen substances or portions (including fats and other tissue portions than a collagen portion) to be removed from the fish skin, while simultaneously degreasing and deodorizing the skin under the salt effect; a salt removal step for causing the admixed salt and non-collagen substances or portions to remove from the skin, achieving the fats removal and deodorization; a collagen extraction step for extracting a collagen (gelatinous) from the thus-treated skin; and a filtration step for filtering and refining the extracted collagen so as to obtain a refined collagen.

Collagen as natural protein polymers finds wide applications in various areas such as medicine and surgery, biochemistry, neuroscience, agriculture, material science, environmental science, chemical engineering, and pharmacology, collagen exhibit great flexibility. As a biomaterial collagen can be modified by scientists into collagen sponges for various biomedical applications, including neuronal regeneration, bone regeneration, and wound healing. Collagen can also form injectable hydrogels or serve as delivery vehicles due to its innate ability to gel with temperature change and is amenable to chemical modifications. (See Eng. Biol., 2017, Vol. 1, Iss. 1, pp. 18-23).

Despite the wide applicability of collagen and its commercial importance, collagen extraction at commercial scale is still challenging and less explored. The extensive and complex extraction process involved in collagen extraction, high cost associated with obtaining pure collagen, high bioburden, long processing time, low yield is some of the hurdles in obtaining collagen.

Therefore, there is a need to explore new and efficient extraction process which provides solution to the above problems associated with known methods such as high bioburden, long processing time, low yield and at the same time, cost effective and easy to scale for industrial applications.

### OBJECTIVE OF THE INVENTION

The main objective of the present invention is to provide a method of producing collagen from an animal skin using a chemical solution and salt additives.

Yet another objective of the present invention is to provide a method for producing collagen from an animal skin where the synergy between the acid in conjunction with metal fluoride results in reduction of bio burden.

Yet another objective of the present invention is to provide a method for producing collagen from an animal skin using a chemical solution and salt additives with protease enzymes reduces the incubation time.

Yet another objective of the present invention is to provide a method where acidic environment created by use of acid increases antimicrobial activity, alters protease activity, releasing oxygen, and reducing toxicity of bacterial end products.

Yet another objective of the present invention is to provide a method for producing collagen where bioburden is better contained and hence method can be operated at higher temperatures.

Yet another objective of the preset invention is to produce nano collagen by using nano milling and liquid nitrogen.

Still another objective of the present invention is to a method for producing collagen which is inexpensive and offers ease of operation.

### SUMMARY OF THE INVENTION

As will be realized in the following description, the invention is capable of other and different embodiments and its several details are capable of modifications in various respects, all without departing from the scope of the present invention.

In one aspect, the present invention relates to a method of producing collagen from an animal skin comprising;
(a) applying to said skin a chemical solution and salt additives and incubating said skin for a predefined sufficient time to loosen attached wool or hair; and
(b) extracting said collagen from said skin.
wherein said solution is a mixture of an organic acid, sodium acetate and water in a predefined ratio and said salt additives are inorganic acid or metal fluorides.

In another aspect, the present invention provides collagen for medical and biotechnology products useful for general surgery, dermatology, dentistry, plastic and reconstructive surgery, neurosurgery, orthopedics, ophthalmology urology, vascular surgery, veterinary medicine, Cosmetics, organ printing, stem cell culture media and other related fields.

Preferably, the collagen is native soluble collagen and finely dispersed native collagen.

Preferably the collagen is collagen nanoparticles prepared using nano-milling.

It will be appreciated by those skilled in the art that the methods disclosed herein may be useful for extracting collagen from any animal skin. However, the present invention is particularly useful for extracting collagen from animal skins taken from animals from the mammalian orders Artiodactyla, Lagomorpha, Rodentia, Perissodactyla, Carnivora and Marsupialia. Preferably, the animal skin is removed from an animal selected from the group consisting of ovine, bovine, caprine, equine, porcine and marsupial. Most preferably, the skin is a sheep skin.

The organic acid in the chemical solution is selected from citric acid, acetic acid, boric acid, alginic acids, preferably acetic acid. The inorganic acid of the salt additive is boric acid and metal fluoride is selected from alkali and alkaline metal fluorides, preferably the metal fluoride is sodium fluoride. The predefined ratio of organic acid, water and sodium in the chemical mixture is 1:1:0.5. The salt additives used are 1.4-2% of the acid present in the chemical mixture.

Once the acid and salt additives has been applied to the animal skin it is allowed to incubate for sufficient time to enable the wool or hair to be loosened or removed. Preferably, the incubation is undertaken at a temperature between 30-40° C. and time between 18-24 hours.

The animal skin may then be chopped or minced into small pieces.

Preferably, the chopped or minced skin is then mixed with chemical mixture in a suspension under non-denaturing conditions and incubated at 0-30 degrees, with stirring. Preferably, the skin is also incubated in the presence of one or more protease enzymes. Preferably, the protease is selected from the group consisting of bromelain and/or Acid fungal protease (AFP). The collagen execrated from protease AFP qualifies as a halal product.

After incubation of the skin pieces in the chemical mixture and protease suspension, the suspension is centrifuged and/or filtered and the soluble and/or dispersed collagen is removed.

The collagen may be dried and ground to form a powder. An alternate to drying and grinding to form a powder, the collagen can be redispersed with the chemical mixture and salt additives, and this dispersion can then be used to form a collagen matrix or sponge by freeze-drying in the manner disclosed in the Silyer et al. U.S. Pat. No. 4,703,108.

In another aspect, the present invention provides a method for producing nano collagen using nano milling and liquid nitrogen that induce nano-milling in collagen manufacturing to improve collagen synthesis.

It is further contemplated that the purified collagen may be comprised of additional compounds, including but not limited to antimicrobials, antivirals, growth factors, anti-dehydration compounds, antiseptics, or other compounds suitable for biomedical and/or veterinary uses.

### BRIEF DESCRIPTION OF THE DRAWINGS

This invention will be described by way of non-limiting embodiments of the present invention, with reference to the accompanying drawings, in which:

FIG. 1 illustrates the TEM images of nano collagen produced using homogenization at 1 ug/mL;

FIG. 2 illustrates the TEM images of nano collagen produced using homogenization and micro fluidization at 1 ug/mL;

FIG. 3 illustrates the TEM images of nano collagen produced using homogenization and micro fluidization at 0.5 ug/mL; and

FIG. 4 illustrates the SDS-PAGE Gel Picture of the Collagen produced according to the present invention.

### DETAILED DESCRIPTION OF THE INVENTION

In the following description, reference is made to the accompanying drawings where, by way of illustration, specific embodiments of the invention are shown. It is to be understood that other embodiments may be used, and other changes may be made without departing from the scope of the present invention. Accordingly, the drawings and detailed description are to be regarded as illustrative in nature and not as restrictive.

Before the present methods are described, it is understood that this invention is not limited to the particular materials and methods described, as these may vary. It is also to be understood that the terminology used herein is for the purpose of describing particular embodiments only and is not intended to limit the scope of the present invention which will be limited only by the appended claims. It must be noted that as used herein and in the appended claims, the singular forms “a,” “an,” and “the” include plural reference unless the context clearly dictates otherwise. Thus, for example, a reference to “an organic acid” is a reference to one or more organic acids and equivalents thereof known to those skilled in the art, and so forth. Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

In one aspect, the present invention relates to a method of producing collagen from an animal skin comprising;
(c) applying to said skin a chemical solution and salt additives and incubating said skin for a predefined sufficient time to loosen attached wool or hair; and
(d) extracting said collagen from said skin.
wherein said chemical solution is a mixture of an organic acid, sodium acetate and water in a predefined ratio and said salt additives are inorganic acid or metal fluorides.

The organic acid in the chemical solution is selected from citric acid, acetic acid, boric acid, alginic acids, preferably acetic acid. The inorganic acid of the salt additive is boric acid and metal fluoride is selected from alkali and alkaline metal fluorides, preferably the metal fluoride is sodium fluoride. The predefined ratio of organic acid, water and sodium acetate in the chemical solution is 1:1:0.5. The salt additives used are 1.4-2% of the chemical solution.

Once the chemical solution and salt additives has been applied to the animal skin it is allowed to incubate for sufficient time to enable the wool or hair to be loosened or removed. Preferably, the incubation is undertaken at a temperature between 30-40° C. and time between 18-24 hours.

In another aspect, the present invention provides a method of producing purified native collagen from an animal skin comprising;
a). providing an animal skin;
b). contacting said skin with chemical mixture and salt additives for predetermined time to substantially digest the glycosaminoglycans present in the skin to produce a pelt;
c). mincing said pelt and exposing to one or more protease(s) for a predetermined time period and temperature to produce a preparation comprising soluble and dispersed collagen;
d). centrifuging or paddle separating said preparation of step (c) to isolate soluble native collagen from dispersed collagen; and
e). precipitating said soluble native collagen with salt to produce purified collagen.

The contacting of animal skin with the chemical mixture causes loosening of the attached skin and wool for step b. The treatment with the chemical mixture and salt additives cause reduction in bioburden to produce endotoxin free collagen and also reduce the dewooling time. The predetermined time to allow the digestion of the glycosaminoglycans present in the skin to produce a pelt is between 18-24 hours and the predetermined time period and temperature for step (c) is between 0-30° C. for 5 to 10 days.

In another aspect, yields of collagen are increased for nano processing by using nano milling and liquid nitrogen. Introducing nano-milling in collagen manufacturing improves collagen synthesis and utilization through the use of nano milling technology. Nano milling involves the reduction of particle size to the nanometer range using mechanical milling techniques. This process can result in various benefits for collagen-based products, including improved collagen dispersion: Collagen is a protein with a large molecular size, and achieving a uniform dispersion of collagen particles in a liquid or gel matrix can be challenging. Nano milling can be used to reduce the size of collagen particles to the nanometer range, resulting in improved dispersion and distribution of collagen in cosmetic formulations or tissue engineering scaffolds.

Nano milling is achieved by any of the process known in the art like homogenization, micro fluidization,
The present invention provides a method for producing nano collagen from an animal skin comprising
a) applying to said skin a chemical mixture and salt additives in a predefined ratio and incubating said skin for a predefined sufficient time to loosen attached wool or hair; and
b) extracting said collagen from said skin,
c) preparing collagen nanoparticles using nano-milling and liquid nitrogen.
wherein said chemical solution is a mixture of an organic acid, sodium acetate and water in a predefined ratio and said salt additives are inorganic acid or metal fluorides

In yet another aspect, the present invention provides a method for producing collagen from an animal skin comprising
washing a mammalian animal skin in detergent and sodium metabisulphite solutions
applying to said skin a chemical solution and salt additives and incubating said skin for sufficient time to loosen attached wool or hair; and
extracting said collagen from said skin.

In yet another aspect, the present invention provides a method for producing collagen from an animal skin comprising dewooling a mammalian pelt using a dewooling paint, said paint comprising acetic acid, anhydrous sodium acetate
applying to said skin a chemical solution and salt additives and incubating said skin for sufficient time to loosen attached wool or hair; and
extracting said collagen from said skin.

In yet another aspect, the present invention provides a method for producing hydrolysable collagen from an animal skin comprising;
a). washing dewooled ovine pelts followed by mincing;
b) digesting minced pelts with citric acid (0.8 g/kg pelt), at pH 2.5-3.5, for 3 to 20 hours at 50 degrees Celsius to obtain acid digested mince;
c) the acid digested mince of step (b) is treated with enzyme mix (mince: enzyme mix-1000:1) at a temperature of 80° C.-100° C. for 5-20 minutes (here enzyme mix is AFP plus bromelain in a ratio of 1:20);
d) insoluble material recovered after enzyme treatment is removed by filtration, the filtrate is spray dried to form collagen powder

Herein, “comprising” means the term “comprising” and certain ingredients are defined as “consisting of” and “consisting essentially of”.

Herein “about” will be understood by persons of ordinary skill in the art and will vary to some extent on the context in which the term is used, “about” will mean up to plus or minus of the particular-term pharmaceutically acceptable ranges.

The invention is described in detail herein below with respect to the following examples, which are provided merely for illustration and are not intended to restrict scope of invention in any manner. Any embodiment that is apparent to a person skilled in the art is deemed to fall within the scope of present invention. It should be understood, however, that the examples following are illustrative only, and should not be taken in any way as a restriction on the generality of the invention described above. For example, while the majority of the examples relate to sheep skins, it is to be understood that the invention can also be applied to other animal skins as disclosed herein, including for example, bovine, porcine and marsupial skins.

### Advantages of Present Invention

1) The process employed by the present invention leads to reduced bioburden; and decreased incubation time and fasts the processing.
2) Chemical solution and salt additives increase utilized by the present invention provides antimicrobial activity, alters protease activity, releasing oxygen and reduces toxicity of bacterial end products
3) Yields are increased for nano processing by using nano milling and liquid nitrogen.
4) Labor cost is reduced

### WORKING EXAMPLES

### Example 1: Method of Producing Collagen from Animal Skin

Chemical solution (1:1:0.5 ratio of acetic acid, water and sodium acetate) and salt additives (1.4% of the chemical solution) are applied to animal skin and incubated at a temperature of between 3° and 40° C. for between 18 and 24 hours time to loosen attached wool or hair. The wool was removed by hand and trimmed to remove any skin containing residual hair or wool to produce a pelt, which was then minced and then introduced to a nondenaturing solution comprising 0.1 M acetic acid, and AFP to obtain a suspension. The suspension was then incubated for 1-7 days at 0-30° C., with stirring. After incubation the suspension was stirred and circulated for mix. The supernatant, containing the solubilized collagen and the precipitate containing the insoluble dispersed collagen were recovered.

### Example 2: Preparation of Purified Native Collagen from Animal Skin (TYPE I and Type III Collagen from a Sheep Skin)

A fresh merino sheepskin with approximately length wool was fleshed mechanically and then sprayed with approximately 50 mL of the chemical solution. The chemical solution used is in the ratio acetic acid:water:sodium acetate in 1:1:0.5 and salt additives sodium fluoride and boric acid as 1.4% of the chemical solution. The skin was incubated at a temperature between 30-40° C. and time between 18-24 hours. The wool was removed by hand and trimmed to remove any skin containing residual hair or wool. The resulting pelt weighed approximately 1.2 kg

The pelt was then washed with water and chopped into small pieces using an industrial grade food processor. The chopped pelt was then introduced to a nondenaturing solution comprising 0.1 M acetic acid, and AFP. The ratio of pelt to non-denaturing solution used was Acid 50:1 raw pelt, AFP 1 kg skins: 0.8 g was then added to the suspension at a ratio of AFP to tissue of 1:100. The suspension was then incubated for 5-10 days at 0-30° C., with stirring. After incubation the suspension was centrifuged, paddle separated to obtain a supernatant. The supernatant, containing the solubilized collagen and the precipitate containing the insoluble dispersed collagen were recovered. The soluble collagen was then precipitated by addition of salt (NaCl) and the precipitate was retained and the supernatant discarded. The precipitate was re-dissolved in dilute (0.1 M) acetic acid, and solution was then centrifuged. The dissolved collagen was dialyzed against several changes of water until a desired level of salt concentration was obtained and then freeze-dried.

### Example 3: Preparation of Collagen Nanoparticles Using Nano-Milling

The collagen is produced as per the procedure of example 1 and nano processing is performed by using nano milling and liquid nitrogen). The temperature of the collagen is decreased between 4 degree Celsius to 20 degree throughout the whole process (so as to liquid state closest to freezing point) followed by running through micro channel high pressure homogenizer at 30-60 thousand pounds per square inch and pass it a second time if need be to maintain batch to batch consistency. The processed collagen is mixed lipoprotein and passed through high shear homogenizer to achieve size of 5 microns (bi-phase lipid in water-based collagen) and again passed through the high-pressure homogenizer to get the droplets go back to 100 nanometers. The back pressure in every batch of high-pressure homogenization is kept between 0 to 25 pounds per square inch to make sure that all the product should be a free-flowing liquid and not a spray (mist).

Sample of nano collagen, produced by different techniques at different concentrations was analyzed by TEM

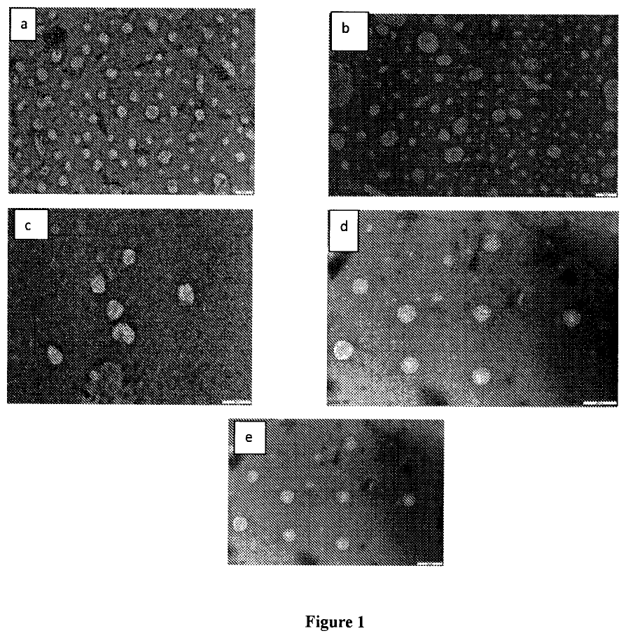

Referring to FIGS. 1, 2 and 3 is illustrated the TEM analysis of nano collagen produced using different process. Nano collagen produced by homogenization, and homogenization with micro fluidization was analyzed by TEM. FIG. 1(a-e) provides the TEM image for nano collagen produced using homogenization at concentration of 1 mg/mL. As it can be seen from the FIG. 1(a-e) that the particle size for the nano collagen is between 45-110 nm and proper encapsulation is not found.

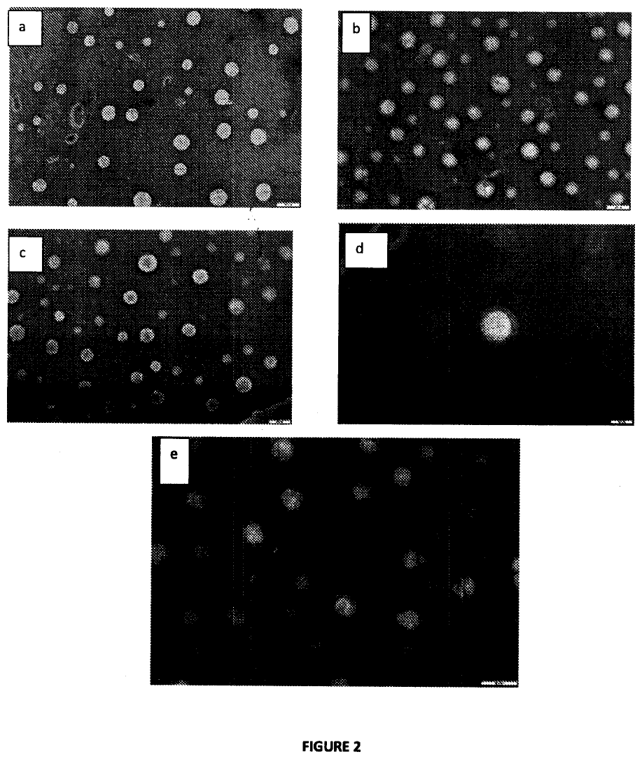

FIG. 2(a-e) the TEM image for nano collagen produced using Homogenization and Microfluidisation at concentration of 1 mg/mL. As it can be seen from the FIG. 2(a-e) that the particle size for the nano collagen is between 75-174 nm and proper encapsulation is seen.

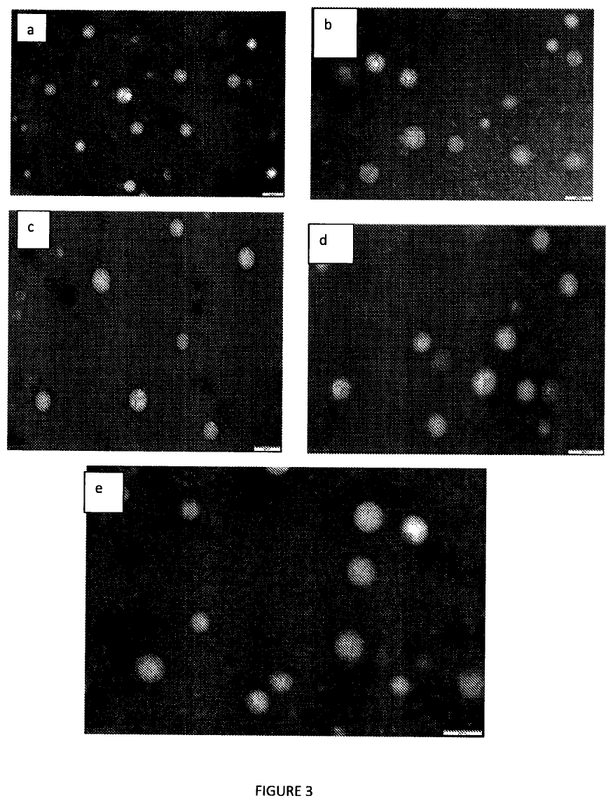

FIG. 3(a-e) the TEM image for nano collagen produced using homogenization at concentration of 0.5 mg/mL. As it can be seen from the FIG. 3(a-e) that the particle size for the nano collagen is between 90-176 nm and proper encapsulation is seen

### Example 4: Preparation of Collagen from a Mammalian Animal Using Dewooling Paint

For the production of collagen ovine pelts were dewooled, and then washed. They are minced and then digested with the combination of acetic acid, (50 L: 1 kg pelt) with AFP eyyzme (1.6 g/kg pelt), at pH 2.5-3.5, for 5-10 days, preferably 7 days. Insoluble material is removed by centrifugation/paddle separation and the protein is salt precipitated. The protein is re-solubilised in the combination of acetic acid, then salt precipitated again. The precipitates are used to produce pure collagen is performed by filtration.

Tables 1˜4 show the composition analysis for the study. Table 1 shows the minced pelts after washing, Table 2 the undigested solids, Table 3 the primary salt precipitation filtrates and Table 4 the products.

TABLE 1
Composition analysis for the minced pelts

| | Total | Protein | Protein | Total | |
|---|---|---|---|---|---|
| Description | solids | NF6.25 | NF5.55 | Fat | Ash |
| mince for Fletchers paint pelt #1 | 26.7 | 12.7 | 11.3 | 13.6 | 0.18 |
| mince for dewooling paint pelt | 18.0 | 13.2 | 11.7 | 3.8 | 0.23 |
| of present invention #1 | | | | | |
| mince for thickened dewooling | 19.8 | 17.2 | 15.3 | 2.2 | 0.26 |
| paint pelt of present invention | | | | | |
| #1 | | | | | |
| mince for Fletchers paint pelt #2 | 19.2 | 12.8 | 11.4 | 4.6 | 0.19 |
| mince for dewooling paint pelt | 18.6 | 14.4 | 12.8 | 4.3 | 0.54 |
| of present invention #2 | | | | | |
| mince for thickened dewooling | 18.9 | 15.1 | 13.4 | 4.4 | 0.27 |
| paint pelt of present invention | | | | | |
| #2 | | | | | |

TABLE 2
Composition analysis for the undigested solids

| | Total | Protein | Protein | Total | |
|---|---|---|---|---|---|
| Description | solids | NF6.25 | NF5.55 | Fat | Ash |
| Undigested solids for Fletchers | 2.6 | 1.8 | 1.6 | 0.57 | <0.1 |
| paint pelt #1 | | | | | |
| Undigested solids for dewooling | 1.8 | 1.5 | 1.3 | <0.2 | <0.1 |
| paint pelt of present invention | | | | | |
| #1 | | | | | |
| Undigested solids for thickened | 3.4 | 3.3 | 2.9 | 0.32 | <0.1 |
| dewooling paint pelt of present | | | | | |
| invention #1 | | | | | |
| Undigested solids for Fletchers | 2.8 | 1.6 | 1.4 | 0.50 | <0.1 |
| paint pelt #2 | | | | | |
| Undigested solids for dewooling | 2.6 | 2.0 | 1.8 | 0.52 | <0.1 |
| paint pelt of present invention | | | | | |
| #2 | | | | | |
| Undigested solids for thickened | 2.1 | 2.0 | 1.8 | 0.52 | <0.1 |
| dewooling paint pelt of present | | | | | |
| invention #2 | | | | | |

TABLE 3
Composition analysis for the salt precipitation filtrates

| | Total | Protein | Protein | Total | |
|---|---|---|---|---|---|
| Description | solids | NF6.25 | NF5.55 | Fat | Ash |
| Filtrate from 1° salt pptn for | 4.3 | <0.1 | <0.1 | <0.2 | 4.2 |
| Fletchers paint pelt #1 | | | | | |
| Filtrate from 1° salt pptn for | 4.4 | <0.1 | <0.1 | <0.2 | 4.2 |
| dewooling paint pelt of present | | | | | |
| invention pelt #1 | | | | | |
| Filtrate from 1° salt pptn for | 4.3 | <0.1 | <0.1 | <0.2 | 4.2 |
| thickened dewooling paint pelt | | | | | |
| of present invention #1 | | | | | |

As there was no detectable protein in the filtrates, they were not analysed for experiment #2.

TABLE 4
Composition analysis for the products

| | Total | Protein | Protein | Total | |
|---|---|---|---|---|---|
| Description | solids | NF6.25 | NF5.55 | Fat | Ash |
| Product from Fletchers paint | 2.5 | 0.90 | 0.79 | 0.69 | 0.90 |
| pelt #1 | | | | | |
| Product from dewooling paint | 2.6 | 1.1 | 0.98 | 0.58 | 1.2 |
| pelt of present invention #1 | | | | | |
| Product from thickened | 3.0 | 1.3 | 1.2 | 0.27 | 1.7 |
| dewooling paint pelt of present | | | | | |
| invention #1 | | | | | |
| Product from Fletchers paint | 2.7 | 0.96 | 0.85 | 0.41 | 1.3 |
| pelt #2 | | | | | |
| Product from dewooling paint | 3.4 | 1.2 | 1.1 | <0.2 | 1.9 |
| pelt of present invention #2 | | | | | |
| Product from thickened | 3.5 | 1.4 | 1.2 | 0.33 | 1.8 |
| dewooling paint pelt of present | | | | | |
| invention #2 | | | | | |

Table 5 shows the Proteomics Results for the products.

TABLE 5
Protein analysis results for products

| | | | | | | Monomer/ |
|---|---|---|---|---|---|---|
| | | | | Type III | | dimer// |
| | | α- | Type | (by dif- | Purity by | trimer/ |
| | Protein | chain | I | ference) | Collagenase | high mass |
| Material | ug/ml | ratio | % | % | % | Ratio |
| Product | 1650 | 2.0* | 82* | 18 | >99 | 42:18: |
| from | 1050 | | | | | 17:23 |
| Fletchers | | | | | | |
| paint pelt | | | | | | |
| #1 | | | | | | |
| Product | 1750 | 1.9* | 77* | 23 | 94 | 43:33:17:8 |
| from | 1700 | | | | | |
| dewooling | | | | | | |
| paint pelt | | | | | | |
| of present | | | | | | |
| invention | | | | | | |
| #1 | | | | | | |
| Product | 2100 | 1.9* | 82* | 18 | 94 | 39:31: |
| from | 1200 | | | | | 17:12 |
| thickened | | | | | | |
| dewooling | | | | | | |
| paint pelt | | | | | | |
| of present | | | | | | |
| invention | | | | | | |
| #1 | | | | | | |
| Product | 1400 | 2.1 | 79 | 21 | >99 | ND |
| from | | | | | | |
| Fletchers | | | | | | |
| paint pelt | | | | | | |
| #2 | | | | | | |
| Product | 1550 | 1.9 | 68 | 32 | 96 | ND |
| from | | | | | | |
| dewooling | | | | | | |
| paint pelt | | | | | | |
| of present | | | | | | |
| invention | | | | | | |
| #2 | | | | | | |
| Product | 1400 | 1.9 | 71 | 29 | >99 | ND |
| from | | | | | | |
| thickened | | | | | | |
| dewooling | | | | | | |
| paint pelt | | | | | | |
| of present | | | | | | |
| invention | | | | | | |
| #2 | | | | | | |

### Example 5: Method for Producing Collagen from an Animal Skin in Detergent and Sodium Metabisulphite Solutions

For the production of collagen ovine pelts were dewooled, and then washed in detergent, sodium metabisulphite solutions and water. The washed pelts are then are minced and then digested with the combination of acetic acid, (50 L: 1 kg pelt) with AFP (1.6 g/kg pelt), at pH 2.5-3.5, for 7 days. Insoluble material is removed by centrifugation and the protein is salt precipitated. The protein is re-solubilised in the combination of acetic acid, and then salt precipitated again, recovered by centrifugation.

### Example 6: Preparation of Hydrolysable Collagen

For the production of hydrolysable collagen ovine pelts were dewooled, and then washed. They are minced and then digested with the combination of citric (0.8 g/kg pelt), at pH 2.5-3.5, for 3 to 20 hours at 50 degrees Celsius to obtain acid digested mince. The acid digested mince is treated with enzyme mix (mince: enzyme mix-1000:1) at a temperature of 80° C.-100° C. for 5-20 minutes (here enzyme mix is AFP plus bromelain in a ratio of 1:20). Insoluble material recovered after enzyme treatment is removed by filtration, the filtrate is spray dried to form collagen powder.

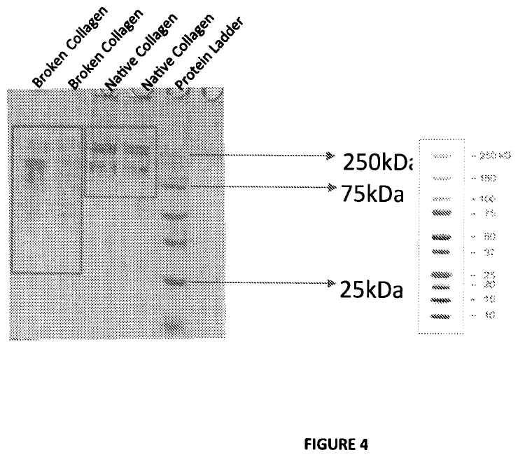

Referring to FIG. 4, is illustrated the SDS-PAGE gel picture of the collagen. Here Green box indicates unprocessed Ovicoll Collagen (120 kDa-300 kDa) and red box indicates processed/milled collagen (35 kDa-300 kDa). This SDS-PAGE is an electrophoresis method that allows protein separation by mass. This allows to characterize the various sizes of collagen entities and their distribution by mass.

It can be concluded that the composition of the products and starting material was somewhat variable but not related to the type of dewooling paint used. The protein content of the mince and products appeared to be higher, and the fat content possibly slightly lower, in the materials dewooled with dewooling paint pelt of present invention.

From the foregoing, it will be appreciated that specific embodiments of the invention have been described herein for purposes of illustration, but that various modifications may be made without deviating from the scope of the invention. Accordingly, the invention is not limited except as by the appended claims.

1. A method for producing collagen from an animal skin comprising:
applying to said skin a chemical solution and salt additives and incubating said skin for a predefined sufficient time to loosen attached wool or hair; and
extracting said collagen from said skin,
wherein said chemical solution is a mixture of an organic acid, sodium acetate and water in a predefined ratio and said salt additives are inorganic acid or metal fluorides.

2. The method according to claim 1, wherein said organic acid is selected from citric acid, acetic acid, boric acid, and alginic acids.

3. The method according to claim 1, wherein the predefined ratio of organic acid, water and sodium acetate in the chemical solution is 1:1:0.5.

4. The method according to claim 1, wherein, the salt additives in the chemical solution are 1.4-2% of chemical solution.

5. The method according to claim 1, wherein the inorganic acid is boric acid.

6. The method according to claim 1, wherein the metal fluoride is selected from alkali and alkaline metal fluorides, preferably the metal fluoride is sodium fluoride.

7. The method according to claim 1, further comprising addition of protease enzymes.

8. The method according to claim 7, wherein the proteases enzymes selected from bromelain and/acid fungal protease (AFP).

9. The method according to claim 1, wherein incubating said skin is performed at a temperature of between 3° and 40° C. for between 18 and 24 hours.

10. A method for producing nano collagen from an animal skin comprising:
applying to said skin a chemical mixture and salt additives in a predefined ratio and incubating said skin for a predefined sufficient time to loosen attached wool or hair;
extracting said collagen from said skin; and
preparing collagen nanoparticles using nano-milling and liquid nitrogen,
wherein said chemical solution is a mixture of an organic acid, sodium acetate and water in a predefined ratio and said salt additives are inorganic acid or metal fluorides.

11. The method according to claim 10, wherein the particle size of the nano collagen is between 45-166 nM.

12. The method according to claim 10, wherein the nano milling is achieved by homogenization, micro fluidization or both.

13. A method for producing collagen from an animal skin comprising:
washing a mammalian animal skin in detergent and sodium metabisulphite solutions;
applying to said skin a chemical solution and salt additives and incubating said skin for sufficient time to loosen attached wool or hair; and
extracting said collagen from said skin.

14. A method for producing collagen from an animal skin comprising:
dewooling a mammalian pelt using a dewooling paint, said paint including comprising acetic acid, anhydrous sodium acetate, sodium fluoride and boric acid;
applying to said skin a chemical solution, salt additive;
incubating said skin for sufficient time to loosen attached wool or hair; and
extracting said collagen from said skin.

15. A method of producing purified native collagen from an animal skin comprising:
providing an animal skin;
contacting said skin with chemical solution and salt additives for predetermined time to substantially digest the glycosaminoglycans present in the skin to produce a pelt;
mincing said pelt and exposing to one or more protease(s) for a predetermined time and temperature to produce a preparation comprising soluble and dispersed collagen;
centrifuging/paddle separation said preparation to isolate soluble native collagen from dispersed collagen; and
precipitating said soluble native collagen with salt to produce purified collagen.

16. The method according to claim 15, wherein:
the predetermined time to allow the digestion of the glycosaminoglycans present in the skin to produce a pelt is between 18-24 hours; and
the predetermined time period and temperature for step (c) is between 0-300 C for 5 to 10 days.

17. The method according to claim 15, wherein the protease is selected from bromelain and/or acid fungal protease (AFP).

18. A method for producing hydrolysable collagen from an animal skin comprising:
washing dewooled ovine pelts followed by mincing;
digesting minced pelts with citric acid (0.8 g/kg pelt), at pH 2.5-3.5, for 3 to 20 hours at approximately 50 degrees Celsius to obtain acid digested mince;
treating the digested minced pelts with enzyme mix at a temperature of 800 C-1000 C for 5-20 minutes, wherein the enzyme mix is AFP plus bromelain in a ratio of 1:20;
removing by filtration an insoluble material recovered after enzyme treatment; and
spray drying the filtrate to form collagen powder.

19. A collagen obtained by the method of claim 1.

20. A nano collagen obtained by the method of claim 10.

21. A collagen obtained by the method of claim 13.

22. A collagen obtained by the method of claim 14.

23. A purified native collagen obtained by the method of claim 15.

24. A hydrolysable collagen obtained by the method of claim 18.
